(19) United States
(12) Patent Application Publication
(10) Pub. No.: US 2014/0358219 A1
(43) Pub. Date: Dec. 4, 2014

(54) Minimally invasive direct Right Internal thoracic artery harvesting and multivessel total arterial CABG using Bilateral internal thoracic arteries through a left minithoracotomy without robotic or thoracoscopic assistance - “The Nambiar Technique”

(71) Applicant: PRADEEP NAMBIAR, Gurgaon (IN)

(72) Inventor: PRADEEP NAMBIAR, Gurgaon (IN)

(21) Appl. No.: 13/769,323

(22) Filed: May 29, 2013

(51) Int. Cl. A61F 2/24; A61B 19/00

(52) U.S. Cl. CPC A61F 2/2427; A61B 19/2203; A61B 2019/2219
USPC 623/1.23

(57) ABSTRACT

Harvesting of Bilateral internal thoracic arteries under direct vision, through a 2 inch left minithoracotomy, without robotic/thoracoscopic assistance and then using these arteries as a composite Y conduit for coronary artery bypass grafting has never been done or described before. The aim was to develop a technique, where bilateral internal thoracic arteries are harvested directly under vision via a 2 inch left minithoracotomy, without robotic/thoracoscopic assistance and complete off pump revascularization of the myocardium done using the internal thoracic arteries as a composite Y conduit.

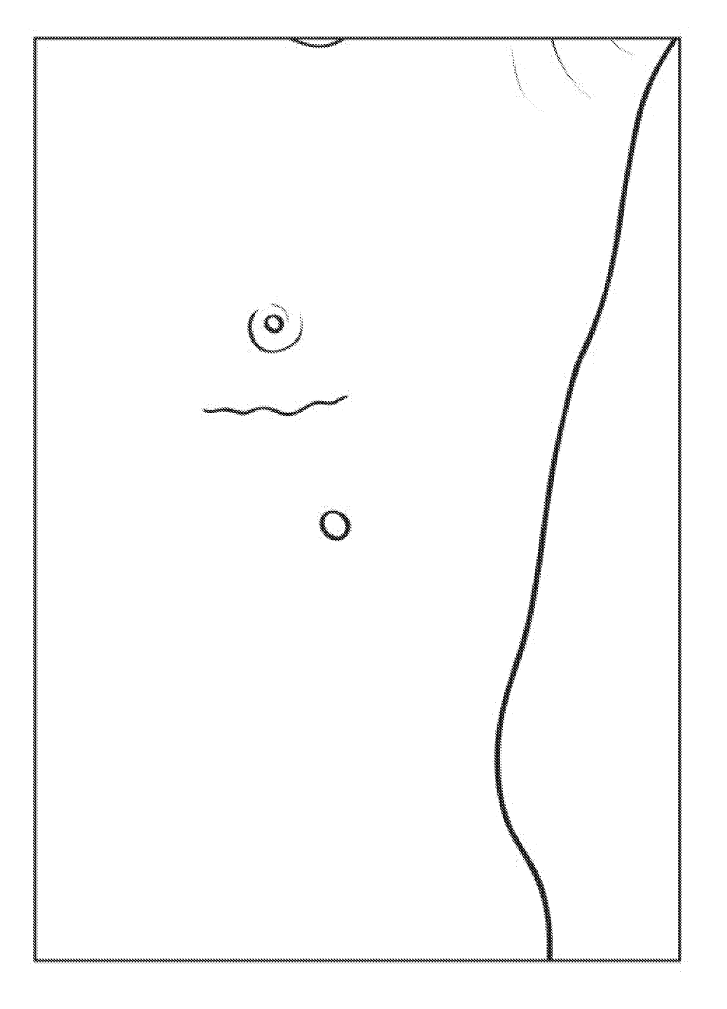

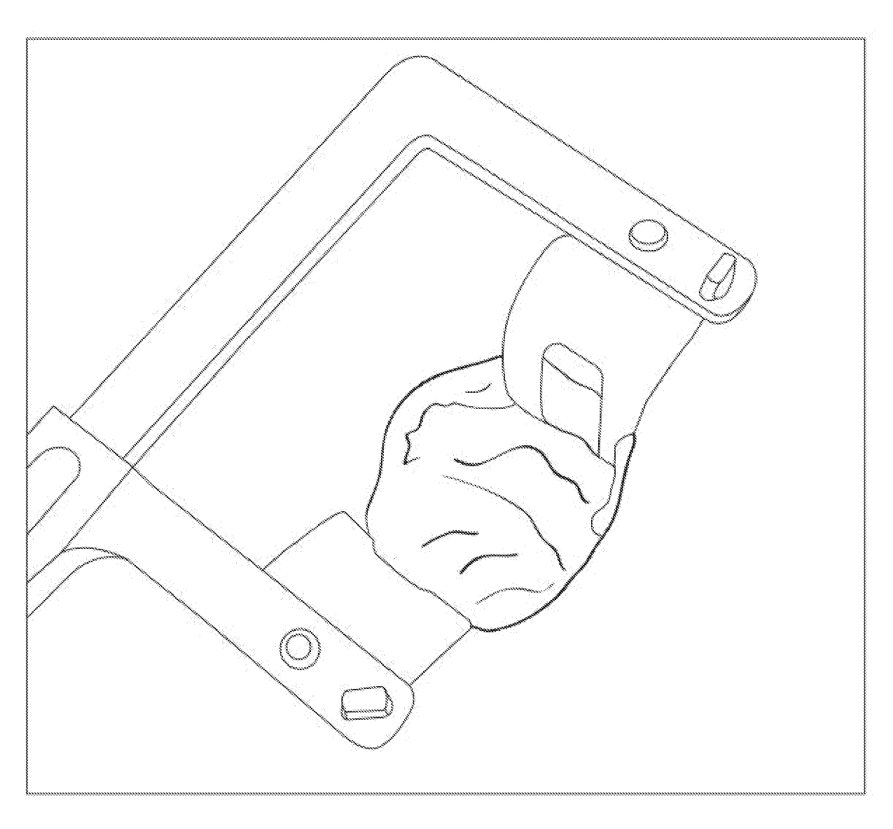

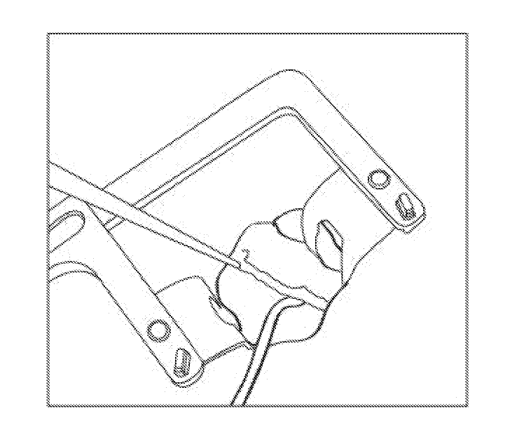

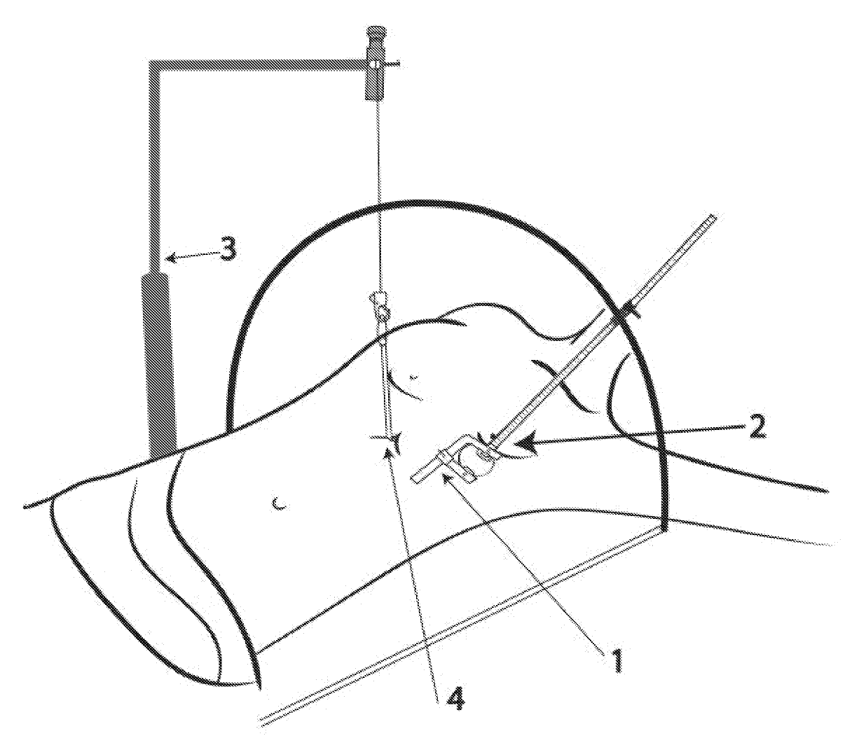

The “Nambiar Technique” which has never been done or described before, encompasses using a 2 inch left minithoracotomy through which the bilateral internal thoracic arteries are harvested directly under vision and multivessel total arterial coronary artery bypass grafting done using these conduits. This technique is reproducible, grossly reduces the invasiveness, does not need infrastructure and will revolutionize coronary artery bypass grafting

The present disclosure generally relates to the medical field and more particularly to the Minimally Invasive technique of Coronary artery bypass grafting through a left minthoracotomy using only Bilateral Internal Thoracic arteries harvested directly under vision without robotic or thoracoscopic assistance. This method has never been done or described before. Use of Bilateral Internal Thoracic arteries in Coronary artery bypass grafting has shown improved survival and increased freedom from reintervention however their usage and the usage of minimal access coronary artery bypass grafting techniques despite all its advantages have not been optimal. The Nambiar Technique encompasses usage of total arterial grafting and minimal access coronary surgery without robotic or thoracoscopic assistance.

Direct harvesting of the Right Internal thoracic artery under vision through a left minithoracotomy, without robotic or thoracoscopic assistance has never been done or described before. The “Nambiar Technique ” encompasses, harvesting of Bilateral Internal Thoracic arteries directly under vision through a 2 inch left minithoracotomy without robotic or thoracoscopic assistance and using the harvested bilateral internal thoracic arteries, as a Left internal thoracic—Right internal thoracic artery composite Y conduit for total arterial revascularization of the myocardium by the off pump methodology. The aim of this procedure, was to bring together and also show feasibility of harvesting bilateral internal thoracic arteries directly under vision, total arterial grafting (Bilateral internal thoracic arteries), minimally invasive coronary surgery, avoidance of sternotomy, reproducibility and excellent outcomes.

Use of Bilateral Internal Thoracic arteries in Coronary artery bypass grafting has shown improved survival and increased freedom from reintervention. Total arterial revascularization with composite arterial grafts has clearly improved the midterm and long term outcomes. However, the usage of Bilateral internal thoracic arteries have not been optimal in Coronary artery bypass grafting because of increased incidence of sternal complications especially in diabetics. Harvesting of Internal thoracic arteries with conventional thoracoscopic instruments with video assist has been limited because of lack of precision, instrument factors and limitations

The introduction of robots have added to the total endoscopic harvest of the Internal thoracic arteries however the limitations have been the cost factor, availability and steep learning curve.

The “Nambiar Technique” which has never been done or described before, encompasses a method in which the internal thoracic arteries are conveniently harvested in a skeletonised manner under direct vision, thereby enabling adequate conduit length for myocardial revascularization. Total arterial revascularization is done using the composite Right internal thoracic artery—Left internal thoracic artery Y conduit by the off pump methodology through a 2 inch″ left minithoracotomy grossly reducing the invasiveness when compared to Coronary artery bypass grafting through a sternotomy. The post operative recovery is excellent with majority of the patients being extubated on the table, operating room times are comparable to standard Coronary artery bypass grafting, post op pain is well controlled with paravertebral block, minimal hospital stay, early discharge as early as 2 days, return to work within 10 days of surgery and excellent financial benefits for both the patient and the hospital. The early outcomes have been excellent and coronary angiograms carried out showed widely patent grafts. This technique is reproducible does not need infrastructure and can be done on an empty beating heart to aid in training.

1. Left minithoracotomy

2. LITA-RITA Y

3. Healed incision

4. Set up/Positioning of the Patient/Minimally Invasive Coronary Surgery Instruments & Rultract retractor for harvesting the Bilateral Internal Thoracic arteries. The instruments have been numbered. (1. Minimally Invasive Coronary Surgery—Coronary Artery Bypass Grafting intercostal retractor. 2. Thorac-Pro Internal Thoracic Artery retractor. 3. Rultract retractor. 4. Rultract retractor elevating lower end of sternum via a sub-xiphoid incision.)

Heart bypass surgery creates a new route, called a bypass, for blood and oxygen to reach your heart. Use of Bilateral Internal Thoracic Arteries in Coronary Artery Bypass Grafting has shown improved survival and increased freedom from reintervention [1]. Total arterial revascularization with composite arterial grafts has clearly improved the midterm and long term outcomes [2]. However, the usage of Bilateral Internal Thoracic Arteries have not been optimal in Cornary Artery Bypass Grafting because of increased incidence of sternal complications especially in diabetics. Harvesting of ITA'S with conventional thoracoscopic instruments with video assist has been limited because of lack of precision, instrument factors and limitations [3]. The introduction of robots have added to the total endoscopic harvest of the ITA'S however the limitations have been the cost factor, availability and steep learning curve. [4]

The “Nambiar Technique” encompasses a method in which the internal thoracic arteries are conveniently harvested in a skeletonised manner under direct vision, thereby enabling adequate conduit length for myocardial revascularization. Total arterial revascularization is done using the composite RITA-LITA Y conduit by the off pump methodology through a 2 inch″ left minithoracotomy grossly reducing the invasiveness when compared to CABG through a sternotomy. The post operative recovery is excellent with majority of the patients being extubated on the table, operating room times are comparable to standard CABG, post op pain is well controlled with paravertebral block, minimal hospital stay, early discharge as early as 2 days, return to work within 10 days of surgery and excellent financial benefits for both the patient and the hospital. The early outcomes have been excellent and coronary angiograms carried out showed widely patent grafts. This technique is reproducible does not need infrastructure and can be done on an empty beating heart to aid in training.

Minimally Invasive Coronary Artery Bypass (MICABG) is a surgical treatment for coronary heart disease that is a less invasive method of coronary artery bypass surgery (CABG). Minimally Invasive coronary surgery is referred to as “keyhole” heart surgery because the operation is analogous to operating through a keyhole.

MICABG is a form of off-pump coronary artery bypass surgery (OPCAB), performed “off-pump”—without the use of cardiopulmonary bypass (the heart-lung machine). MICABG differs from OPCAB in the type of incision used for the surgery; with traditional CABG and OPCAB a median sternotomy (dividing the breastbone) provides access to the heart; with MICABG, the surgeon enters the chest cavity through a left mini-thoracotomy (a 2 inch incision between the ribs).

The patients are placed in a supine position, with slight elevation of the left chest to about 30 degrees. The non dominant arm with normal modified Allen's test is abducted to ninety degrees and placed on an arm support. (in case the radial artery is required)

The patients are intubated with a double lumen endotracheal tube for single lung ventilation and standard invasive monitoring with arterial line, pulmonary artery catheter and trans esophageal echo are done.

Surface marking of the right and left internal thoracic arteries are done using a vascular Doppler and skin marking pencil.

A 2 inch left inframammary incision is made two finger breadths lateral to the Left internal thoracic artery surface marking and the thoracic cavity is entered through the 5th intercostal space. Using a minimal access intercostal retractor [Fehling Inc] in the 5th intercostal space the ribs are gently spread. The pericardium is then opened in an inverted T fashion and the coronary arteries are inspected following which the pericardiotomy is closed with interrupted 2-0 silk sutures.

A thorac-pro Internal Thoracic Artery (Fehling Inc; Germany) retractor is then used in tandem with the minimal access intercostal retractor and the chest is elevated. The left hemi thorax is thoroughly inspected and flow in the Left internal thoracic artery is studied with a vascular Doppler. The fatty attachments between the pericardium and the sternum are completely divided and on dissecting the pleural from the endothoracic fascia of the right chest wall the Right internal thoracic artery is well visualized.

For enhanced visualization of the lower thirds and beyond the bifurcation, a 0.5 inch sub xiphoid incision is made and a langenbeck retractor insinuated on the undersurface of the sternum. Traction is then given via a Rultract Internal thoracic artery retractor, thereby elevating the lower third of the sternum, which greatly enhances visualization of the distal end of the Right Internal Thoracic artery. This incision is later used to insert a pleural drain.

The right pleura is widely opened as this helps in positioning the circumflex vessels for grafting without any hemodynamic compromise. Using a bovie at a very low setting, the Right Internal Thoracic Artery is harvested in a skeletonised fashion from the subclavian vein proximally to the bifurcation distally. The Right Internal Thoracic artery length is more than adequate to reach the Right Coronary artery and right coronary artery—posterior descending artery. The Left Internal Thoracic artery is then harvested in a standard fashion.

Following heparinisation a Left Internal Thoracic artery—Right Internal Thoracic artery Y composite conduit is constructed and this is used for complete myocardial revascularization by the Off Pump coronary artery bypass technique technique using only the Guidant Acrobat coronary artery stabilizer. Positioning of the heart is further aided by traction sutures on the pericardial edges and by rotation of the operating table. The Left Internal Thoracic artery is anastomosed to the Left anterior descending artery and the Right Internal Thoracic artery Y is used for sequential grafting of the circumflex and inferior wall vessels.

Majority of the patients are extubated on the table and are mobilised within an hour of return from the operating room. Analgesia is optimized using the continuous paravertebral block technique with an infusion of 0.25% sensoricaine. All monitoring lines and chest drains are removed on the first post-op morning. Majority of the patients are discharged on the 2nd or 3 post-op day.

### REFERENCES

1. Lytle B W, Blackstone E H, Loop F D. Two internal thoracic arteries are better than one. J Thorac Cardiovasc. Surg. 1999; 117: 855-72

2. Muneretto C, Negri A, Manfredi J. Safety and usefulness of composite grafts for total arterial myocardial revascularization: A prospective randomized evaluation. J Thorac Cardiovasc. Surg. 2003; 125: 826-835

3. Subramanian V, Patel N, Patel N. Robotic assisted multivessel minimally invasive direct coronary artery bypass with port access stabilization and cardiac positioning: Paving the way for outpatient coronary surgery. Ann. Thoracic Surg; 2005; 79:1590-96.

4. Jones B, Desai P, Poston R. Establishing the case for minimally invasive, robotic assisted CABG in the treatment of multivessel coronary artery disease. Heart surg Forum; 2009 June; 12(3) E 147-149.

This study indicates that Coronary artery bypass grafting using bilateral internal thoracic arteries can be conveniently done through a 2 inch left minithoracotomy without robotic or thoracoscopic assistance. Further, this technique has never been described or done before. This technique also brings together the gold standard of total arterial grafting using internal thoracic arteries and minimal invasive coronary surgery

The description of the invention including its applications and advantages as set forth herein is illustrative and is not intended to limit the scope of the invention, which is set forth in the claims. These and other variations and modifications of the embodiments disclosed herein, including of the alternatives and equivalents of the various elements of the embodiments, may be made without departing from the scope and spirit of the invention.

1. A technique of Coronary artery bypass grafting which has never been done or described before and where the entire operation is carried out through a 2 inch left mini thoracotomy.

2. The system of claim 1, further comprising of minimal invasive coronary artery bypass grafting being done using only bilateral internal thoracic arteries.

3. The system of claim 2, further comprising of the bilateral internal thoracic arteries harvested under direct vision without any robotic or thoracoscopic assistance, this has never been done or described.

4. The system of claim 3, further comprising the harvested Internal thoracic arteries are constructed into a Y composite conduit for use in total arterial myocardial revascularization.

5. The system of claim 4, wherein the Internal thoracic artery Y composite conduit is used for total myocardial revascularization using the off pump technique.

6. The system of claim 4, where the Internal thoracic arteries are used as sequential grafts for revascularisation

7. The system of claim 6, wherein the internal thoracic arteries are harvested directly under vision without any robotic or thoracoscopic assistance and the entire revascularization is done without any endoscopic assistance.

8. The system of claim 7, wherein the internal mammary arteries are harvested using the skeletonised technique, thereby ensuring better flow and longer coduits for the composite Y

9. The system of claim 8, whereby no sternotomy is carried out and hence ensuring less mortality and morbidity

10. The system of claim 9, ensuring better cosmesis, increased survival, minimal pain, elimination of blood transfusion and infection.

11. The system of claim 10, ensuring earlier discharge from hospital and return to normal activity.

12. The system of claim 11, wherein minimal access surgery is carried out avoiding the use of robot or videoscopes thereby reducing costs and being beneficial to both the patient and the hospital
